(19) United States
(12) Patent Application Publication
(10) Pub. No.: US 2011/0033382 A1
(43) Pub. Date: Feb. 10, 2011

(54) Imaging Agents

(75) Inventor: Mark M. Goodman, Atlanta, GA (US)

Correspondence Address: Patent Administrator;Office of Technology Transfer, Emory University, 1599 Clifton Road NE, 4th Floor, Atlanta, GA, 30322, US

(73) Assignee: EMORY UNIVERSITY, Atlanta, GA (US)

(21) Appl. No.: 12/937,323

(22) PCT Filed: Apr. 9, 2009
(86) PCT No.: PCT/US09/39997

§ 371 Date: Oct. 11, 2010

Related U.S. Application Data
(60) Provisional application No. 61/044,725, filed on Apr. 14, 2008.

(51) Int. Cl. A61K 51/04; C07C 61/06

(52) U.S. Cl. 424/1.89; 562/504

(57) ABSTRACT

This invention provides amino acid derivatives useful in detecting and evaluating brain and body tumors, including (1S,2S) anti-2-[18F]FACPC and (1R,2R) anti-2-[18F]FACPC.

This application claims the benefit of U.S. Provisional Application No. 61/044,725, filed Apr. 14, 2008, which is incorporated herein in its entirety to the extent not inconsistent herewith.

### BACKGROUND OF THE INVENTION

This invention generally relates to amino acid analogs having specific and selective binding in a biological system, particularly brain and systemic tumors, and capable of being used for positron emission tomography (PET) and single photon emission (SPECT) imaging methods.

The development of radiolabeled amino acids for use as metabolic tracers to image tumors using positron emission tomography (PET) and single photon emission computed tomography (SPECT) has been underway for some time. PET and SPECT are particularly useful imaging techniques for brain tumors. After surgical resection and/or radiotherapy of brain tumors, conventional imaging methods such as CT and MRI do not reliably distinguish residual or recurring tumor from tissue injury due to the intervention and are not optimal for monitoring the effectiveness of treatment or detecting tumor recurrence [Buonocore, E (1992), Clinical Positron Emission Tomography. Mosby-Year Book, Inc. St. Louis, Mo., pp 17-22; Langleben, D D et al. (2000), J. Nucl. Med. 41:1861-1867]. Therefore, it is necessary to develop imaging agents useful with PET and SPECT.

The leading PET agent for diagnosis and imaging of neoplasms, 2-[¹⁸F]fluorodeoxyglucose (FDG), has limitations in the imaging of brain tumors. Normal brain cortical tissue shows high [¹⁸F]FDG uptake as does inflammatory tissue which can occur after radiation or surgical therapy; these factors can complicate the interpretation of images acquired with [¹⁸F]FDG [Griffeth, L K et al. (1993), Radiology. 186:37-44; Conti, P S (1995)].

Amino acids are required nutrients for proliferating tumor cells. A number of reports indicate that PET and SPECT imaging with radiolabeled amino acids better define tumor boundaries within normal brain than CT or MRI allowing better planning of treatment [Ogawa, T et al. (1993), Radiology. 186: 45-53; Jager, P L et al. (2001), Nucl. Med., 42:432-445]. Additionally, some studies suggest that the degree of amino acid uptake correlates with tumor grade, which could provide important prognostic information [Jager, P L et al. (2001) J. Nucl. Med. 42:432-445].

Some amino acids containing the positron emitting isotopes carbon-11 and fluorine-18 have been prepared and evaluated for potential use in clinical oncology for tumor imaging in patients with brain and systemic tumors and may have superior characteristics relative to 2-[¹⁸F]FDG in certain cancers. These amino acid candidates can be subdivided into two major categories. The first category is represented by radiolabeled naturally occurring amino acids such as [¹¹C]valine, L-[¹¹C]leucine, L-[¹¹C]methionine (MET) and L-[1-¹¹C]tyrosine, and structurally similar analogues such as 2-[¹⁸F]fluoro-L-tyrosine and 4-[¹⁸F]fluoro-L-phenylalanine. The movement of these amino acids across tumor cell membranes predominantly occurs by carrier mediated transport by the sodium-independent leucine type “L” amino acid transport system. The increased uptake and prolonged retention of these naturally occurring radiolabeled amino acids into tumors in comparison to normal tissue is due in part to significant and rapid regional incorporation into proteins. Of these radiolabeled amino acids, [¹¹C]MET has been most extensively used clinically to detect tumors. Although [¹¹C]MET has been found useful in detecting brain and systemic tumors, it is susceptible to in vivo metabolism through multiple pathways, giving rise to numerous radiolabeled metabolites. Thus, graphical analysis with the necessary accuracy for reliable measurement of tumor metabolic activity is not possible. Studies of kinetic analysis of tumor uptake of [¹¹C]MET in humans strongly suggest that amino acid transport may provide a more sensitive measurement of tumor cell proliferation than protein synthesis.

The shortcomings associated with [¹¹C]MET may be overcome with a second category of amino acids. These are non-natural amino acids such as 1-aminocyclobutane-1-[¹¹C]carboxylic acid ([¹¹C]ACBC). The advantage of [¹¹C]ACBC in comparison to [¹¹C]MET is that [¹¹C]ACBC is not metabolized. However, a significant limitation in the application of carbon-11 amino acids for clinical use is the short 20-minute half-life of carbon-11. The 20-minute half-life requires an on-site particle accelerator for production of the carbon-11 amino acid. In addition, only a single or relatively few doses can be generated from each batch production of the carbon-11 amino acid. Therefore carbon-11 amino acids are poor candidates for regional distribution for widespread clinical use.

In order to overcome the physical half-life limitation of carbon-11, several fluorine-18 labeled non-natural amino acids have been developed. Some of these compounds are disclosed in U.S. Pat. Nos. 5,808,146 and 5,817,776, and WO 03/093412 which are incorporated herein by reference. The primary reasons for proposing ¹⁸F-labeled amino acids analogs instead of ¹¹C (t_{1/2}=20 min.) are the substantial logistical and economic benefits gained with using ¹⁸F instead of ¹¹C-labeled radiopharmaceuticals in clinical applications. The advantage of imaging tumors with ¹⁸F-labeled radiopharmaceuticals in a busy nuclear medicine department is primarily due to the longer half-life of ¹⁸F (t_{1/2}=110 min.). The longer half-life of ¹⁸F allows off-site distribution and multiple doses from a single production lot of radio tracer. In addition, these non-metabolized amino acids may also have wider application as imaging agents for certain systemic solid tumors that do not image well with 2-[¹⁸F]FDG using PET. Some fluorine-18 amino acids can be used to image brain and systemic tumors in vivo based upon amino acid transport with PET.

There is a continued need for novel imaging agents which can bind tumor cells or tissues with high specificity and selectivity and can be readily prepared in sufficient quantities for tumor imaging with PET and SPECT. As a candidate compound makes the transition from validation studies in cells in vitro and animal models to application in humans, the synthetic methods employed must allow routine, reliable production of the compound in large quantities. The present application discloses compounds, methods of synthesizing and using those compounds for tumor imaging with PET and SPECT.

### SUMMARY OF THE INVENTION

The present invention provides novel amino acid compounds useful in detecting and evaluating brain and systemic tumors and other uses.

In one embodiment, compounds of the invention have the following general formula (Formula I):

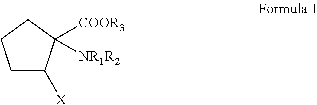

wherein R₁ and R₂ are each independently selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, haloalkynyl, Tc-99m and Re chelates; R₃ is selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, and haloalkynyl;
X is selected from the group consisting of halogen, haloalkyl, halocycloalkyl, halocycloalkenyl, halocycloalkynyl, haloacyl, haloaryl, haloheteroaryl, haloalkenyl, haloalkynyl, Tc-99m chelate and Re chelate, where halo or halogen in X is selected from the group consisting of F, Cl, Br, I, At, F-18, Br-76, I-123, I-124. All positions that are not specified may be hydrogen, or may be substituted independently by a substituent selected from the group consisting of H, alkyl, haloalkyl, cycloalkyl, halocycloalkyl, heteroaryl, aryl, haloaryl, haloheteroaryl, alkenyl, haloalkenyl, alkynyl, haloalkynyl, where halo is non-radioactive F, Cl, Br and I.

In certain embodiments, all of R1, R2 and R3 are hydrogen. In certain embodiments, R3 is hydrogen, and one of R1 and R2 is hydrogen, and the other is C1-C6 alkyl. In certain embodiments, X is radiolabeled. In certain embodiments, X is either F-18, Br-76, I-123 or I-124. In certain embodiments, X is a C1-C6 haloalkyl.

The compounds provided herein are generally referred to as ACPC compounds (1-amino-cyclopentane-1-carboxylic acid).

While the general formulas shown for Formula I do not show the stereochemistry of the substituents, it is intended that every structural isomer and stereoisomer, including all enantiomers, racemates, racemic mixtures, and diastereomers, of the compounds of Formula I are included in this disclosure individually and collectively. These included compounds include all individual stereoisomers of the compounds of Formula I. Some specific compounds provided are: anti-2-[¹⁸F] FACPC; syn-2-[¹⁸F] FACPC; (1R,2R)-(−)-anti-2-[¹⁸F]FACPC); (1S,2S)-(+)-anti-2-[¹⁸F]FACPC); a mixture of (1S,2S) and (1R,2R) anti-1-amino-2-[¹⁸F]fluorocyclopentyl-1-carboxylic acid; (1S,2S) anti-1-amino-2-[¹⁸F]fluorocyclopentyl-1-carboxylic acid; and (1R,2R) anti-1-amino-2-[¹⁸F]fluorocyclopentyl-1-carboxylic acid.

Also provided are synthesis methods for the provided compounds.

The amino acid compounds of the invention bind target tumor tissues or cells with high specificity and selectivity when administered to a subject in vivo. Preferred amino acid compounds show a target to non-target ratio of at least 2:1, are stable in vivo and substantially localized to target within 1 hour after administration. Because of their high specificity and selectivity for tumor tissues, the inventive compounds can also be used in delivering a therapeutic agent to a given tumor site.

Any of F, Cl, Br, I or C in the formulas above may be in stable isotopic or radioisotopic form. Particularly useful radioisotopic labels are ¹⁸F, ¹²³I, ¹²⁵I, ¹³¹I, ⁷⁶Br, ⁷⁷Br and ¹¹C. The compounds of the invention can also be labeled with technetium and rhenium. Technetium-99m is known to be a useful radionuclide for SPECT imaging. The cyclic amino acids of the invention are joined to a Tc-99m metal cluster through a 4-6 carbon chain which can be saturated or possess a double or triple bond. The Tc-99m metal cluster can be, for example, an alkylthiolato complex, a cytectrene or a hydrazino nicotinamide complex (HYNIC). U.S. Pat. No. 5,817,776 describes various methods of synthesizing [Tc-99 m] technetium containing compounds in detail, which is incorporated herein in its entirety.

The inventive compounds labeled with an appropriate radioisotope are useful for tumor imaging with PET and/or SPECT, which can serve as diagnostic purposes or evaluating efficacy of any therapeutic compounds for a given tumor. The inventive method of imaging a tumor comprises (a) introducing into a subject a detectable quantity of a labeled compound disclosed herein such as a compound of Formula I or a pharmaceutically acceptable salt, ester or amide thereof; (b) allowing sufficient time for the labeled compound to become associated with tumor tissue; and (c) detecting the labeled compound associated with the tumor with PET or SPECT.

The present invention also provides diagnostic compositions comprising a radiolabeled compound of Formula I and optionally a pharmaceutically acceptable carrier or diluent. Also within the scope of the invention are pharmaceutical compositions which comprise a compound of Formula I and optionally a pharmaceutically acceptable carrier or diluent. The pharmaceutical compositions are useful for delivering a therapeutic agent to a specific tumor site in a subject.

### BRIEF DESCRIPTION OF THE FIGURES

FIG. 1 shows uptake of the 1st peak enantiomer for control and reference compounds BCH, MeAlB, and ACS in 9L cells. For the control experiments, cells are exposed to the listed compounds for 30 minutes in amino acid free media in the absence of any inhibitor (such as BCH, MeAlB, or ACS).

FIG. 2 shows uptake of the 1st peak enantiomer for control, and compounds BCH, MeAlB, and ACS in DU-145 androgen independent prostate cancer cells.

FIG. 3 shows uptake of the 1st peak enantiomer for control, and compounds BCH, MeAlB, and ACS in A549 lung cancer cells.

FIG. 4 shows uptake of the 1st peak enantiomer for control, and compounds BCH, MeAlB, and ACS in MIA U87 glioma cells.

FIG. 5 shows uptake of the 1st peak enantiomer for control, and compounds BCH, MeAlB, and ACS in MIA PaCa-2 pancreas cancer cells.

FIG. 6 shows uptake of the 1st peak enantiomer for control, and compounds BCH, MeAlB, and ACS in MDA mb 231 breast cancer cells.

FIG. 7 shows uptake of the 1st peak enantiomer for control, and compounds BCH, MeAlB, and ACS in MDA mb 468 breast cancer cells.

FIG. 8 shows uptake of the 1st peak enantiomer for control, and compounds BCH, MeAlB, and ACS in SKOV 3 ovarian cancer cells.

FIG. 9 shows uptake of the 1st peak enantiomer for control, and compounds BCH, MeAlB, and ACS in LnCap androgen dependent cancer cells.

FIG. 10 shows uptake of the 1st peak enantiomer for control, and compounds BCH, MeAlB, and ACS in 9L cells.

FIG. 11 shows uptake of the 2nd peak enantiomer for control, and compounds BCH, MeAlB, and ACS in DU 145 androgen independent prostate cancer cells.

FIG. 12 shows uptake of the 2nd peak enantiomer for control, and compounds BCH, MeAlB, and ACS in A549 lung cancer cells.

FIG. 13 shows uptake of the 2nd peak enantiomer for control, and compounds BCH, MeAlB, and ACS in U 87 glioma cells.

FIG. 14 shows uptake of the 2nd peak enantiomer for control, and compounds BCH, MeAlB, and ACS in MIA PaCa-2 pancreas cancer cells.

FIG. 15 shows uptake of the 2nd peak enantiomer for control, and compounds BCH, MeAlB, and ACS in MDA mb231 breast cancer cells.

FIG. 16 shows uptake of the 2nd peak enantiomer for control, and compounds BCH, MeAlB, and ACS in MDA mb 468 breast cancer cells.

FIG. 17 shows uptake of the 2nd peak enantiomer for control, and compounds BCH, MeAlB, and ACS in SKOV 3 ovarian cancer cells.

FIG. 18 shows uptake of the 2nd peak enantiomer for control, and compounds BCH, MeAlB, and ACS in LnCap androgen dependent cancer cells.

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

The term “pharmaceutically acceptable salt” as used herein refers to those carboxylate salts or acid addition salts of the compounds of the present invention which are suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “pharmaceutically acceptable salt” as used herein in general refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. See, for example, Berge S. M, et al., Pharmaceutical Salts, J. Pharm. Sci. 66:1-19 (1977) which is incorporated herein by reference.

Similarly, the term, “pharmaceutically acceptable carrier,” as used herein, is an organic or inorganic composition which serves as a carrier/stabilizer/diluent of the active ingredient of the present invention in a pharmaceutical or diagnostic composition. In certain cases, the pharmaceutically acceptable carriers are salts. Further examples of pharmaceutically acceptable carriers include but are not limited to water, phosphate-buffered saline, saline, pH controlling agents (e.g. acids, bases, buffers), stabilizers such as ascorbic acid, isotonizing agents (e.g. sodium chloride), aqueous solvents, a detergent (ionic and non-ionic) such as polysorbate or TWEEN 80.

The term “alkyl” as used herein by itself or as part of another group refers to a saturated hydrocarbon which may be linear, branched or cyclic of up to 10 carbons, preferably 6 carbons, more preferably 4 carbons, such as methyl, ethyl, propyl, isopropyl, butyl, t-butyl, and isobutyl. The alkyl groups disclosed herein also include optionally substituted alkyl groups where one or more C atoms in the backbone are replaced with a heteroatom, one or more H atoms are replaced with halogen or —OH. The term “aryl” as employed herein by itself or as part of another group refers to monocyclic or bicyclic aromatic groups containing from 5 to 12 carbons in the ring portion, preferably 6-10 carbons in the ring portion, such as phenyl, naphthyl or tetrahydronaphthyl. Aryl groups may be substituted with one or more alkyl groups which may be linear, branched or cyclic. Aryl groups may also be substituted at ring positions with substituents that do not significantly detrimentally affect the function of the compound or portion of the compound in which it is found. Substituted aryl groups also include those having heterocyclic aromatic rings in which one or more heteroatoms (e.g., N, O or S, optionally with hydrogens or substituents for proper valence) replace one or more carbons in the ring.

“Acyl” group is a group which includes a —CO— group.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as used herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” employed herein by itself or as part of another group refers to chlorine, bromine, fluorine or iodine which may be radiolabeled or not.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazlinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁₋₄ alkyl, C₂₋₄ aminoalkyl, C₁₋₄ halo alkyl, halo benzyl, or R^{a} and R^{b} are taken together to form a 5- to 7-member heterocyclic ring optionally having O, S or NR^{c} in said ring, where R^{c} is hydrogen or C₁₋₄ alkyl.

The compounds of the invention are useful as tumor binding agents and as NMDA receptor-binding ligands, and in radio-isotopic form are especially useful as tracer compounds for tumor imaging techniques, including PET and SPECT imaging. Where X is At, the compounds have utility for radio-therapy. Particularly useful as an imaging agent are those compounds labeled with F-18 since F-18 has a half-life of 110 minutes, which allows sufficient time for incorporation into a radio-labeled tracer, for purification and for administration into a human or animal subject. In addition, facilities more remote from a cyclotron, up to about a 200 mile radius, can make use of F-18 labeled compounds.

SPECT imaging employs isotope tracers that emit high energy photons (γ-emitters). The range of useful isotopes is greater than for PET, but SPECT provides lower three-dimensional resolution. Nevertheless, SPECT is widely used to obtain clinically significant information about analog binding, localization and clearance rates. A useful isotope for SPECT imaging is [¹²³I], a γ-emitter with a 13.3 hour half life. Compounds labeled with [¹²³I] can be shipped up to about 1000 miles from the manufacturing site, or the isotope itself can be transported for on-site synthesis. Eighty-five percent of the isotope's emissions are 159 KeV photons, which is readily measured by SPECT instrumentation currently in use.

Accordingly, the compounds of the invention can be rapidly and efficiently labeled with [¹²³I] for use in SPECT analysis as an alternative to PET imaging. Furthermore, because of the fact that the same compound can be labeled with either isotope, it is possible to compare the results obtained by PET and SPECT using the same tracer.

Other halogen isotopes can serve for PET or SPECT imaging, or for conventional tracer labeling. These include ⁷⁵Br, ⁷⁶Br, ⁷⁷Br and ⁸²Br as having usable half-lives and emission characteristics. In general, the chemical means exist to substitute any halogen moiety for the described isotopes. Therefore, the biochemical or physiological activities of any halogenated homolog of the compounds of the invention are now available for use by those skilled in the art, including stable isotope halogen homologs. Astatine can be substituted for other halogen isotopes, [²¹⁰At] emits alpha particles with a half-life of 8.3 h. At-substituted compounds are therefore useful for tumor therapy, where binding is sufficiently tumor-specific.

The invention provides methods for tumor imaging using PET and SPECT. The methods entail administering to a subject (which can be human or animal, for experimental and/or diagnostic purposes) an image-generating amount of a compound of the invention, labeled with the appropriate isotope and then measuring the distribution of the compound by PET if [¹⁸F] or other positron emitter is employed, or SPECT if [¹²³I] or other gamma emitter is employed. An image-generating amount is that amount which is at least able to provide an image in a PET or SPECT scanner, taking into account the scanner's detection sensitivity and noise level, the age of the isotope, the body size of the subject and route of administration, all such variables being exemplary of those known and accounted for by calculations and measurements known to those skilled in the art without resort to undue experimentation.

It will be understood that compounds of the invention can be labeled with an isotope of any atom or combination of atoms in the structure. While [¹⁸F], [¹²³I] and [¹²⁵I] have been emphasized herein as being particularly useful for PET, SPECT and tracer analysis, other uses are contemplated including those flowing from physiological or pharmacological properties of stable isotope homologs and will be apparent to those skilled in the art.

The compounds of the invention can also be labeled with technetium (Tc) via Tc adducts. Isotopes of Tc, notably Tc^{99m}, have been used for tumor imaging. The present invention provides Tc-complexed adducts of compounds of the invention, which are useful for tumor imaging. The adducts are Tc-coordination complexes joined to the cyclic amino acid by a 4-6 carbon chain which can be saturated or possess a double or triple bond. Where a double bond is present, either E (trans) or Z (cis) isomers can be synthesized, and either isomer can be employed. The inventive compounds labeled with Tc are synthesized by incorporating the ^{99m}Tc isotope as a last step to maximize the useful life of the isotope.

The amino acid compounds of the invention may synthesized in specialized, non-standard routes to maximize a useful lifetime for short-lived isotopes (i.e., last step incorporation of isotopes), and to maximize yield and purity, as described below.

ACPC, also known as cycloleucine, has also been evaluated as a chemotherapeutic agent. [Berlinguet, L., Begin, N., and Sarkar, N. K. (1962). Mechanism of antitumour action of 1-amino-cyclopentane carboxylic acid. Nature 194, 1082-1083. and Carter, S. K. NSC-1026.] A comparative preclinical study of 1-amino-cyclopropane-1-carboxylic acid (ACPRC), ACBC, ACPC and 1-amino-cyclohexne-1-carboxylic acid (ACHC) labeled with carbon-14 was performed in rats transplanted with Morris 51236 hepatomas. [Washburn, L. C., Sun, T. T., Anon, J. B., and Hayes, R. L. (1978). Effect of structure on tumor specificity of alicyclic alpha-amino acids. Cancer Res 38, 2271-2273.] In this study, ACBC and ACPC demonstrated significantly higher tumor uptake (4.6 and 3.6% dose/g), respectively than ACPRC and ACHC (1.2 and 0.9% dose/g, respectively).

[¹¹C]ACPC has been used to a limited extent to evaluate systemic tumors. In a preliminary study using [¹¹C] ACPC in 33 patients with known breast metastases, ameloblastoma, lymphoma and metastatic adenocarcinoma, and lung, breast and bone cancer, increased uptake was observed in 70% of lesions using a single photon rectilinear scanner. (Hubner K F, Andrews G A, Washburn L, Wieland B W, Gibs W B, Hayes R, Butler T A, Winebrenner J D. Tumor Location with 1-Aminocyclopentane-1 [¹¹C]Carboxylic Acid: Preliminary Clinical Trials with Single-Photon Detection, J Nucl Med 1977; 18: 1215-1221).

In a more recent study comparing [¹¹C]ACPC with [¹¹C]ACBC in 7 human subjects with an islet cell tumor, bronchogenic cancer and lung (breast cancer), bone (breast cancer) and liver (lung cancer) metastasis, increased uptake was observed in lesions in all subjects using a PET scanner. [Hubner, K. F., Krauss, S., Washburn, L. C., Gibbs, W. D., and Holloway, E. C. (1981). Tumor detection with 1-aminocyclopentane and 1-aminocyclobutane C-11-carboxylic acid using positron emission computerized tomography. Clin Nucl Med 6, 249-252.]

### Synthesis of (1S,2S) and (1R,2R) anti-[¹⁸F]FACPC

Scheme 1 outlines the preparation of the racemic mixture (1S,2R) and (1R,2S) anti-[¹⁸F]FACPC labeling precursor 9 and its conversion into (1S,2S) and (1R,2R) anti-[¹⁸F]FACPC, 13 and 14, respectively.

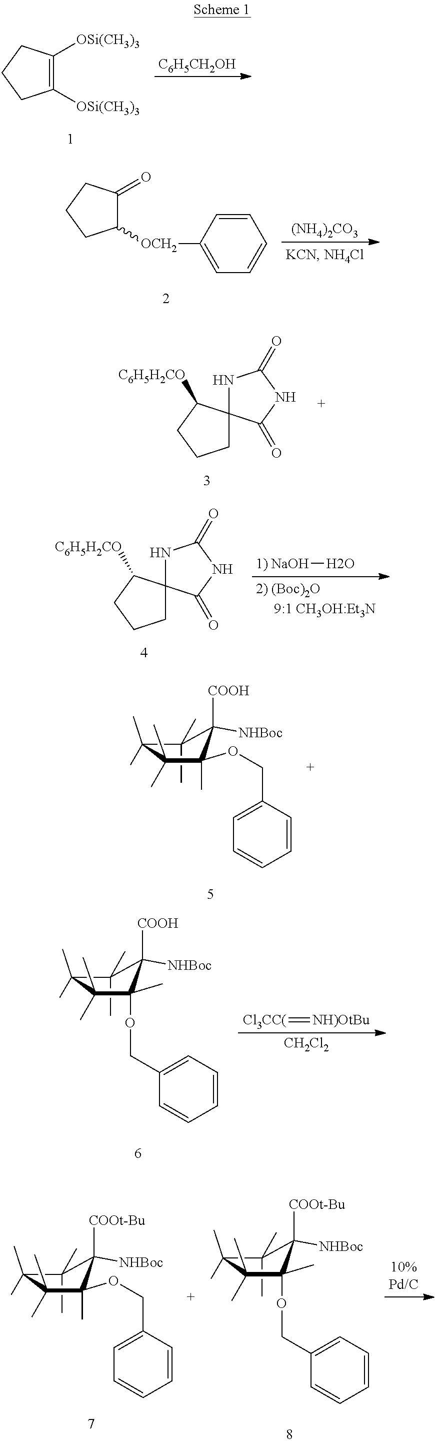

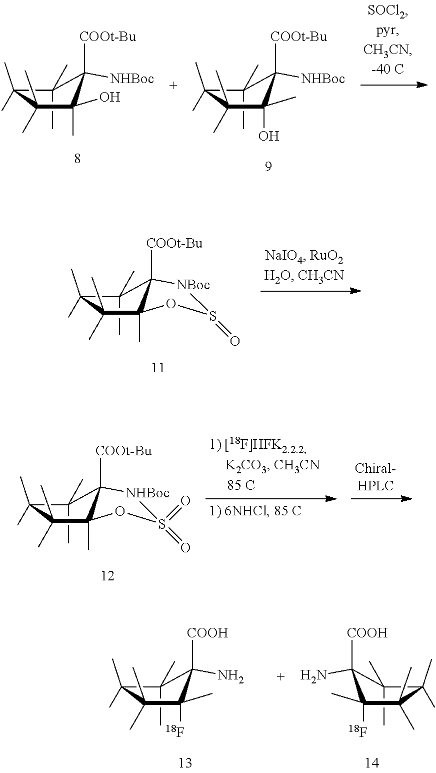

Scheme 2 outlines the stereoselective synthesis of (1S,2S) and (1R,2R) anti-2-[¹⁸F]FACPC, 13 and 14, employing an asymmetric Strecker synthesis.

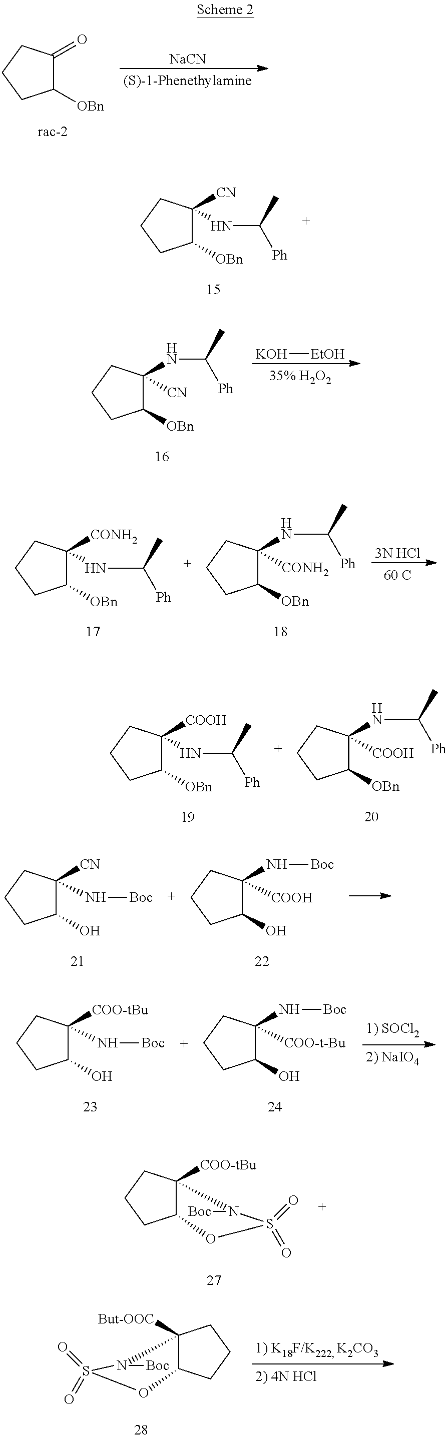

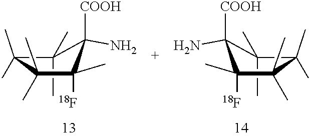

It is noted that in the schemes, the positioned lines on ring carbons are only meant to indicate the position of hydrogen atoms attached to the ring. The lines are not meant to indicate that methyl groups are present, unless specifically labeled as such.

Attachment of a substituent, such as an alkyl group (for example C1-C6 alkyl) onto to the 2-position whereby the radioactive atom is attached to the alkyl group makes possible the preparation of both syn- and anti-compounds. For example, the synthesis outlined above allows preparation of (1S,2S) and (1R,2R) syn- and anti-[¹⁸F]fluoromethylACPC; (1S,2S) and (1R,2R) syn- and anti-[¹⁸F]fluoroethylACPC; (1S,2S) and (1R,2R) syn- and anti-[¹⁸F]fluoropropylACPC, and other fluoroalkyl compounds.

### Example 1

### Synthesis of (1R,2R) and (1S,2S) anti-2-FACPC

syn-5-(2-benzyloxycyclopentane)hydantoin (3) and anti-5-(2-benzyloxycyclopentane)hydantoin (4). To a solution of 4 eq of ammonium carbonate (1.82 g, 18.9 mmoles) and 2 eq of ammonium chloride (0.4 g, 7.56 mmoles) in 13 mL of water was added 1 eq of the cyclopentanone 2 (0.36 g, 1.89 mmoles) in 13 mL of ethanol. After stirring at room temperature for 15 minutes, a 1.2 eq portion of potassium cyanide (0.6 g, 9.45 mmoles) was added, and the reaction mix was heated at 70° C. overnight. The solvent was removed under reduced pressure, and the crude cream colored solid was rinsed thoroughly with water to remove salts. The product (0.2 g, 41%) was obtained as a 9:1 mixture of syn:anti isomers, 5 and 6, respectively.

syn-1-(N-(tert-butoxycarbonyl)amino)-2-benzyloxycyclopentane-1-carboxylic acid (5). A suspension of compounds 3 and 4 (1.2 g, 4.6 mmoles) in 55 mL of 3N sodium hydroxide was heated at 115-120° C. overnight in a sealed stainless steel vessel. After cooling, the reaction mix was neutralized to pH 6-7 with concentrated hydrochloric acid. After evaporation of water under reduced pressure, the resulting solid was extracted with hot ethanol. The combined ethanol extracts were concentrated, and the residue was dissolved in 50 mL of 9:1 methanol:triethylamine. To the solution was added a 1.5 eq portion of di-tert-butyl dicarbonate (2.11 g, 9.68 mmol), and the solution was stirred at room temperature for 15 h. The solvent was removed under reduced pressure, and the crude product was purified by flash chromatography on SiO2 GF with CH₂Cl₂:Et₃N, 9:1. The N-Boc acid 5 (360 mg, 23%) was obtained as a light yellow oil.

syn-1-(N-(tert-butoxycarbonyl)amino)-2-benzyloxycyclopentane-1-carboxylic acid tert-butyl ester (8). A 2.5 eq portion of tert-butyl 2,2,2-trichloroacetamide (1.5 g, 6.9 mmol) was added to a solution of N-Boc acid 5 (360 mg, 1.07 mmoles) in 8 mL of dichloromethane. After 3 days of stirring, the reaction mixture was filtered, washed with dichloromethane and the filtrate concentrated under reduced pressure, and the crude product was purified via silica gel column chromatography (CH₂Cl₂:MeOH, 9:1). The N-Boc tert-butyl ester 8 (230 mg, 55%) was obtained as a colorless oil.

syn-1-(N-(tert-butoxycarbonyl)amino)-3-hydroxycyclopentane-1-carboxylic acid tert-butyl ester (10). To a solution of 8 (100 mg, 0.25 mmoles) in 5 mL of CH₃OH under an argon atmosphere was added 105 mg of 10% Pd/C. The reaction mix was stirred overnight at room temperature under a hydrogen atmosphere. The suspension was then filtered over Celite and concentrated under reduced pressure purification via silica gel column chromatography (CH₂Cl₂:MeOH, 9:1), which provided the alcohol 10 (75 mg, 86%) as a clear oil.

syn-4-(tert-butoxycarbonyl)-2,3,4-oxathiazabicyclo[3.2.0]octane-6-carboxylic acid tert-butyl ester 2-oxide (11). A solution of the N-Boc alcohol 10 (65 mg, 0.22 mmol) was added to a cooled (−40° C.) solution of 2.5 eq of thionyl chloride (65 mg, 40 μL) in 1 mL acetonitrile under an argon atmosphere followed by the addition of 5 eq of pyridine (87 mg, 89 μL) in 1 mL of acetonitrile. After 10 minutes the cooling bath was removed, and the reaction was continued for 30 minutes. The reaction mix was partitioned between 10 mL of EtOAc and 10 mL of H₂O. The aqueous layer was further extracted with 3×10 mL of EtOAc. The organic layers were combined and washed with 20 mL of brine followed by usual work up. Silica gel column chromatography (12.5% EtOAc in hexane) afforded cyclic sulfamidite 11 as a colorless oil (61 mg, 80%).

syn-4-(tert-butoxycarbonyl)-2,3,4-oxathiazabicyclo[3.2.0]octane-6-carboxylic acid tert-butyl ester 2,2-dioxide (12). A solution of the sulfamidite 11 (58 mg, 0.017 mmol) in 7 mL of CH₃CN was cooled in an ice bath and treated successively with 1.1 eq of NalO₄ (41 mg), a catalytic amount of RuO₂.H₂O (˜0.4 mg) and 42 mL of H₂O. After 30 minutes of stirring, the ice bath was removed, and the reaction was continued for 20 minutes. The reaction mixture was diluted in 10 mL of EtOAc and washed with 10 mL of saturated NaHCO₃ solution. The aqueous layer was extracted with 2×10 mL of EtOAc, and the combined organic layers were washed with 10 mL brine followed by usual work up. The crude product was purified by silica gel column chromatography (12% EtOAc in hexane) to provide the cyclic sulfamidate 12 as a clear oil (54 mg, 87%).

Preparation of (1S,2S) and (1R,2R) anti-1-amino-2-[¹⁸F]fluorocyclopentyl-1-carboxylic acid ((1S,2S) and (1R,2R) anti-2-[¹⁸F]FACPC), 13. To a glass vessel containing 1.67 Ci of no-carrier-added [¹⁸F]HF (50 μA, 60 minute bombardment, theoretical specific activity of 1.7 Ci/nmole) in 0.6 mL H₂O containing 5 mg of K₂CO₃ was added a 1 mL solution of 5 mg K₂₂₂ Kryptofix in CH₃CN. The solvent was removed at 110° C. with argon gas flow, and an additional 1 mL of CH₃CN was added followed by evaporation with argon flow. This drying was repeated a total of 3 times to remove residual H₂O. A 2-5 mg portion of the cyclic sulfamidate precursor 12 in 1 mL of dry CH₃CN was added to the vial, and the reaction mix was heated at 90° C. for 10 minutes. The solvent was removed at 115° C. with argon gas flow, and the intermediate product was treated with 0.5 mL of 4N HCl at 110° C. for 10 minutes. The aqueous hydrosylate was allowed to cool for 1 minute and then diluted with approximately 4 mL of sterile saline. The aqueous solution was then transferred to an ion retardation (IR) column assembly consisting of a 7×120 mm bed of AG 11 A8 ion retard resin, a neutral alumina SepPak Plus (preconditioned with 10 mL water) and an HLB Oasis cartridge (preconditioned with 10 mL ethanol then blown dry with 20 mL air), and rinsed with 60 mL of sterile water and then attached to a dose vial. The product [¹⁸F]13 was eluted in series through the ion retard resin, the alumina SepPak Plus and the HLB Oasis cartridge. The elution was performed with three successive portions of ˜4 mL sterile saline transferred from the glass vial to the IR column assembly. The radiolabeled product eluting from the column assembly passed through a 0.22 μm sterile filter into a dose vial.

In all radiosyntheses, the only peak present on radiometric TLC analysis corresponded to 13 and the radiochemical purity of the product exceeded 99%. The isolated radiochemical yield (398 mCi, 38% (decay corrected, non-optimized) was determined using a dose-calibrator (Capintec CRC-712M).

HPLC Separation of (1S,2S) and (1R,2R) anti-1-amino-2-[¹⁸F]fluorocyclopentyl-1-carboxylic acid ((1S,2S) and (1R,2R) anti-2-[¹⁸F]FACPC), 13 50 uL×4 of the mixture was injected onto a Astec Chirobiotic T HPLC column (4.6 mm×250 mm, MeOH) 1 mL/min Enantiomer 1,400 μCi, (Peak 1) Rt=75 sec, Enantiomer 2, 530 μCi, (Peak 2) Rt=90 sec.

Anti-2-[F-18]FACPC exists as two sets of enantiomers (1R,2R) and (1S,2S). The 2 enantiomers were separated on analytical Chirobiotic T and TG columns using methanol as the eluent. The absolute configuration to each of the isolated anti-2-[F-18]FACPC enantiomers has not yet been assigned. Peak #1 corresponds to the enantiomer that elutes first off the column whereas Peak # 2 corresponds to the enantiomer that elutes second.

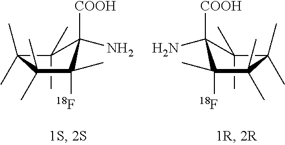

### Example 2

### Tumor Binding Specificity

Compounds have been evaluated in vitro for tumor binding specificity (i.e. uptake cells) using a variety of tumor cell lines available in the art, along with reference compounds such as Me—AlB and BCH. Detailed description of these assays can be found in Martarello et al. (2002) Journal of Medicinal Chemistry, 45:2250-2259 and McConathy et al. (2003) Nuclear Medicine and Biology, 30:477-490. These so called “amino acid uptake studies” are typically carried out with radiolabeled compounds in at least five phenotypically different human tumor cell lines (e.g., A549 lung carcinoma, MB468 breast carcinoma, DU145 prostate carcinoma-androgen independent, LnCap-androgen dependent, SKOV3 ovarian carcinoma, U87 glial blastoma, MIA PaCa-2 pancreas carcinoma, MDA MB231 breast carcinoma). These tumor cell lines can be grown either in vitro or in vivo with severe combined immunodeficiency (SCID) mice as a host. The afore-mentioned tumor cell lines are available at the Winship Cancer Institute of Emory University.

The cancer cell lines evaluated were:

| Name | Description | Name | Description | Name | Description |
|---|---|---|---|---|---|
| A549 | Human Lung | DU145 | Human Prostate | MIA PaCa-2 | Human Pancreas |
| | adenocarcinoma | | carcinoma | | carcinoma |
| 9L | Rat gliosarcoma | U87 | Human Glioma | MDAMB231 | Human Breast |
| | | | | | carcinoma |
| MDAMB468 | Human Breast | SKOV3 | Human Ovarian | LnCap | Human Prostate |
| | carcinoma | | carcinoma | | carcinoma |

For the in vitro amino acid uptake studies, all cells can be grown to monolayer confluency in T-175 culture flasks [Corning, Corning, N.Y.] (approx. 1×10⁸ cells/flask) in Dulbecco's Modified Eagle's Medium (DMEM) [Sigma, St. Louis, Mo.] in a humidified incubator (37° C., 5% CO₂/95% air). Media are supplemented with 10% fetal calf serum [Hyclone, Logan, Utah], and antibiotics (10,000 U/ml penicillin and 10 mg/ml streptomycin) (Sigma, St. Louis, Mo.). For tissue culture passage, monolayer cells are detached by gentle trypsinization, resuspended in complete media, and split 1:10 into new T-flasks. Cultures are passaged weekly, and fed fresh media every 2 to 3 days. To initiate tumor growth in SCID mice, 1×10⁶ cells are injected s.c. bilaterally into the flanks (inguinal region) of the recipient animals using a 1 ml syringe with a 27 gauge needle. Ex vivo experiments can be performed with animals containing tumors weighing between 500 mg and 1 g, as estimated by caliper measurement (tumor weight=(π/6)*abc, where a, b and c are the tumor length, width and height, respectively).

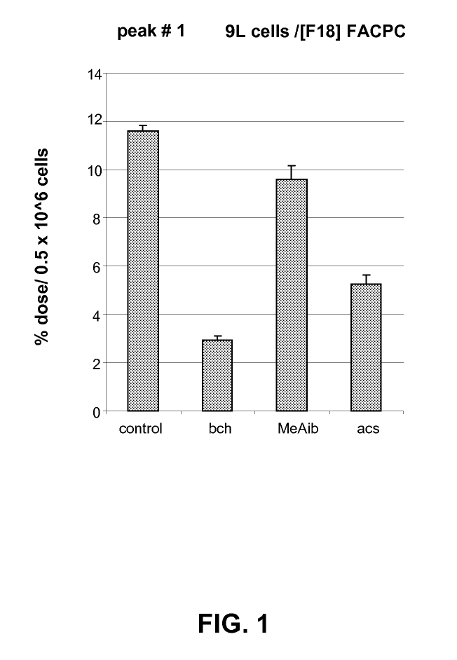

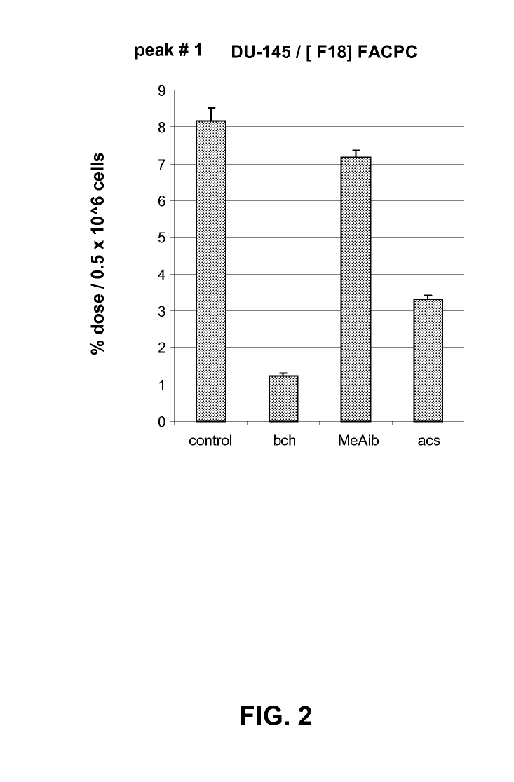

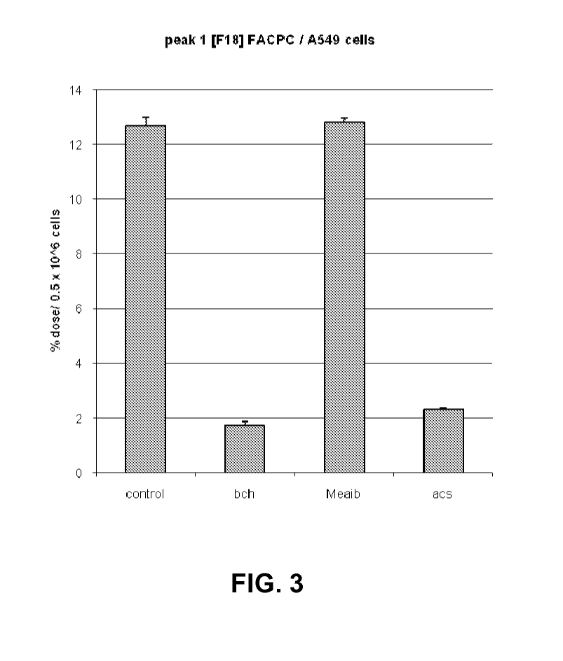

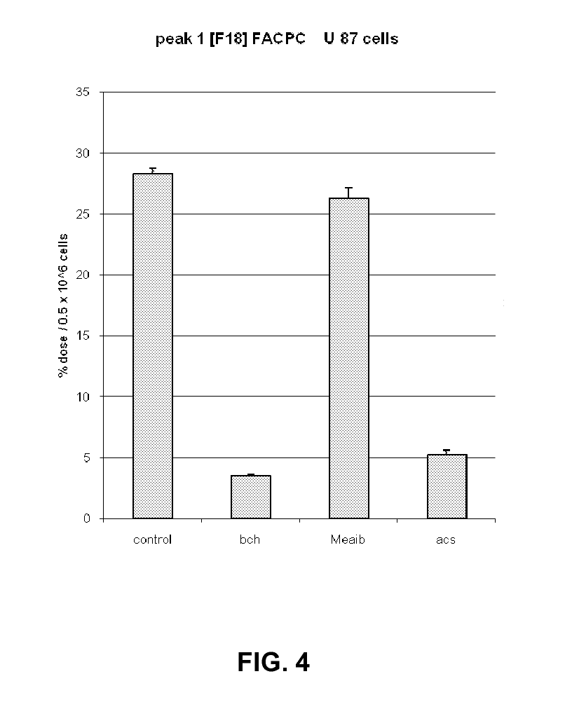

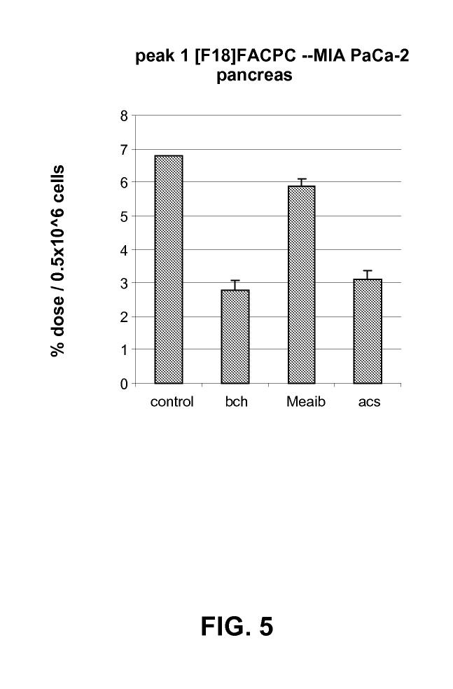

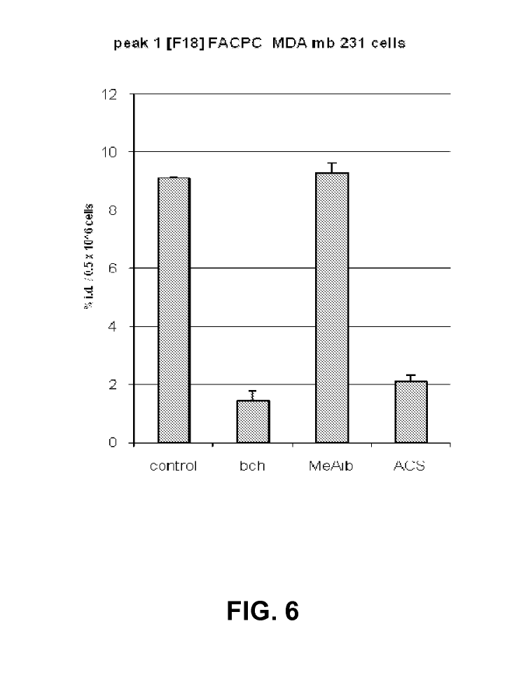

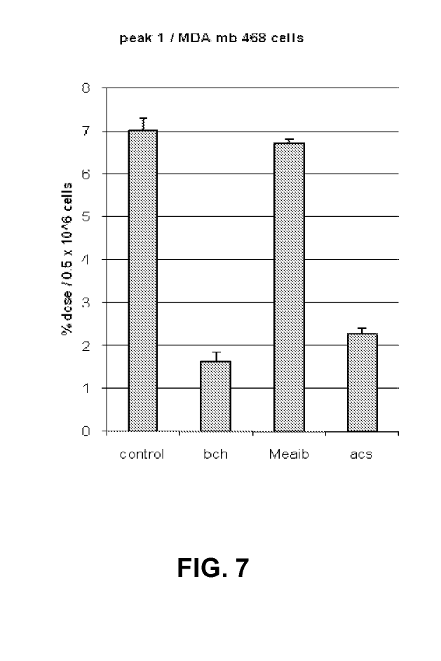

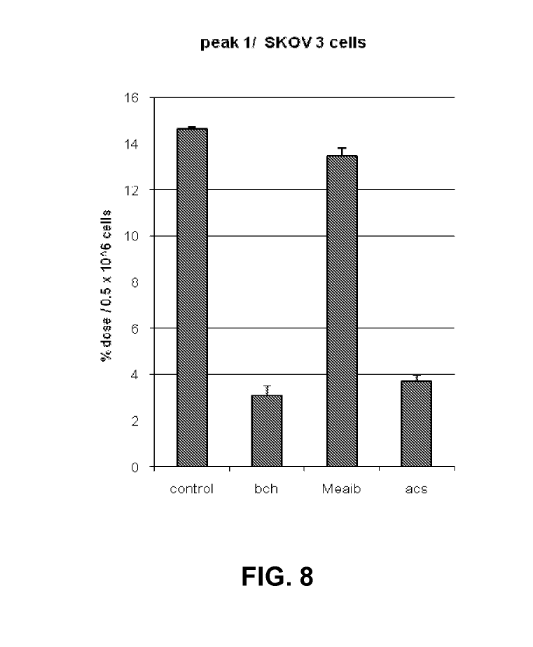

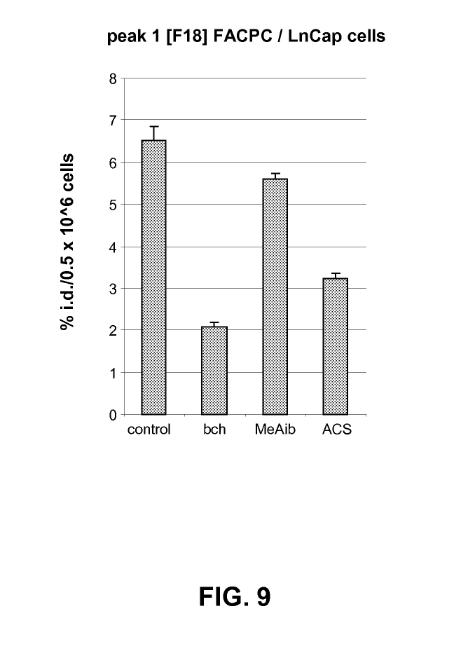

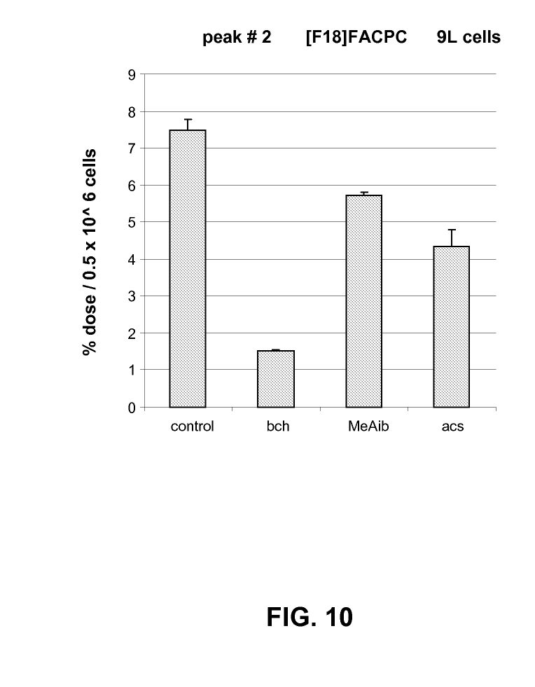

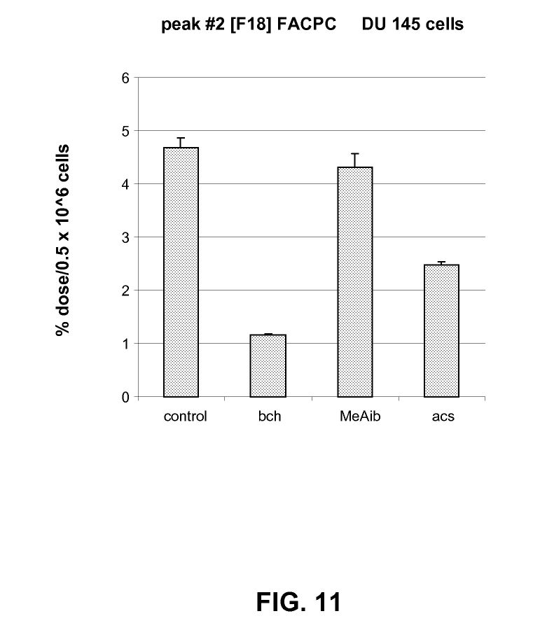

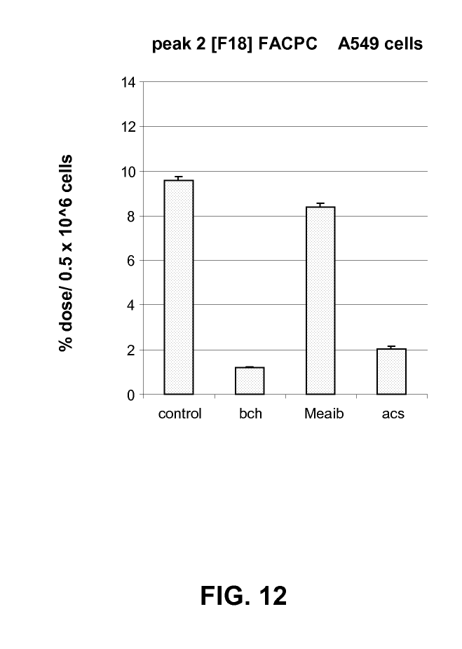

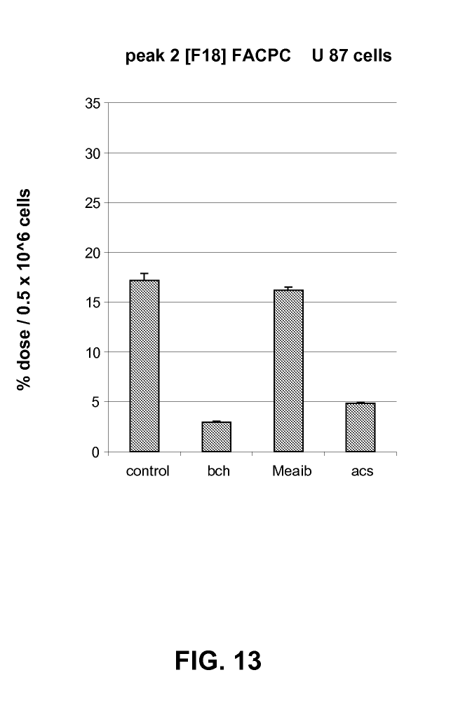

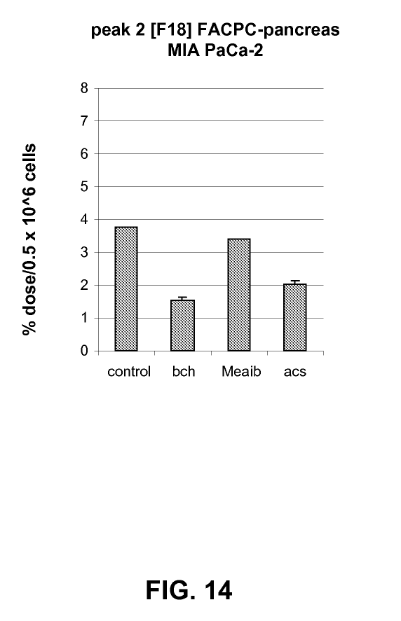

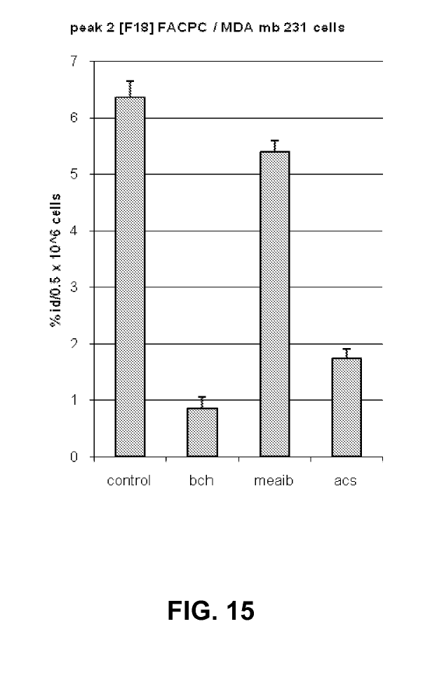

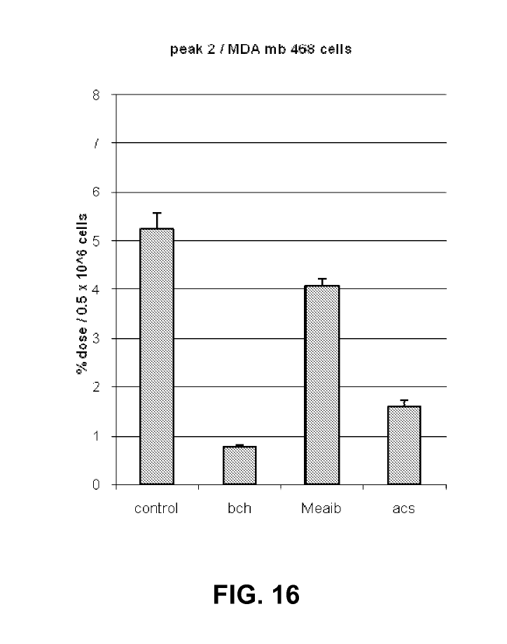

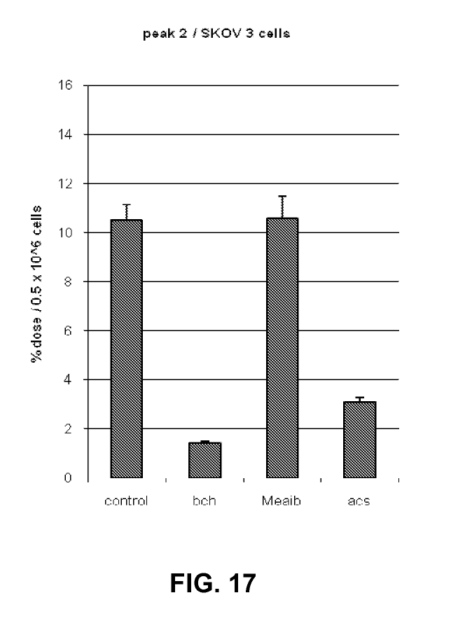

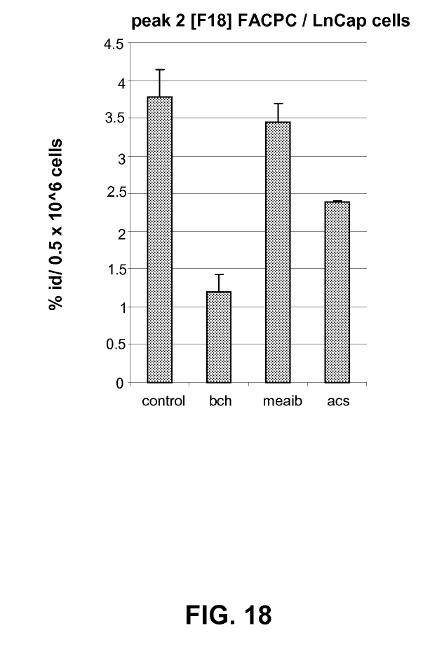

In the in vitro studies the uptake rate of each amino acid compound is measured in each tumor line, as well as the dominant transport mechanisms of each tumor cell line. After trypsinization, cells are resuspended in serum-free media, then counted on a hemocytometer, with viability assessed through Trypan blue staining. Approximately 1×10⁷ cells are exposed to each compound (15 μCi) in 15 ml of amino acid free media for 5, 10, 15, 30 and 60 minutes at 37° C. Cells are then centrifuged at 150×g for 5 minutes, rinsed in 5 ml cold-saline, recentrifuged, resuspended in 3 ml saline, and placed into 12×75 mm glass vials (Fisher, Pittsburgh, Pa.). The vials are placed in a Cobra-II gamma counter (Packard, Meriden, Conn.), with the activity per cell number determined. Inhibition studies determine the dominant transport mechanism (L, A or ASC) for each line [Martarello et al. (2002) supra; McConathy et al. (2003) supra]. For these studies, cells are exposed to the compounds for 30 minutes in amino acid free media containing one of three inhibitors (2-amino-norbornyl-2-carboxylic acid (BCH), 10 mM; α-(methylamino)-isobutyric acid (MeAlB), 10 mM; and an alanine-serine-cysteine mixture 1:1:1 (acs), 10 mM). Saline washes are performed as described above, and the filtered cells' radioactivity determined on the gamma counter. Comparisons with the 30 minute control uptake indicate the major transporters used. Results of these studies are found in FIGS. 1-18.

TABLE 1
% Dose uptake of anti-2-[¹⁸F]FACPC per 0.5 × 10⁻⁶ Cells

| | Enantiomer 1 | Enantiomer 2 |
|---|---|---|
| Cell Line | (% Dose/0.5 × 10⁻⁶) | (% Dose/0.5 × 10⁻⁶) |
| 9L | 12 | 7.5 |
| DU145 | 8 | 4.7 |
| A549 | 13 | 9.6 |
| U87 | 28 | 17 |
| MIA PaCa-2 | 6.8 | 3.8 |
| MDA MB231 | 9.1 | 6.4 |
| SKOV3 | 7 | 5.5 |
| MDA MB468 | 15 | 7.5 |
| LnCap | 6.5 | 3.8 |

TABLE 2
% Dose uptake of racemic anti-2-[¹⁸F]FACBC per 0.5 × 10⁻⁶ Cells

| | Enantiomer 1 |
|---|---|
| Cell Line | (% Dose/0.5 × 10⁻⁶) |
| 9L | 2.3 |
| DU145 | 6.4 |
| A549 | 8.9 |
| U87 | 8.3 |
| SKOV3 | 2.2 |
| MDA MB468 | 7.4 |

The compounds of the invention are further evaluated for their tumor specificity and selectivity in tumor-bearing animal models. One can evaluate and compare the transport, accumulation and tissue distribution of each compound in these in vivo animal studies.

Tissue distribution of the compounds is measured in SCID mice (average weight, 20-25 g) bearing human tumors as follows. The candidate radioligands (20 uCi in 0.4 ml 0.9% NaCl) are injected into the tail vein of tumor-bearing mice. The animals are sacrificed (cervical dislocation) at 5, 30, 60 and 120 minutes post-injection. Tissues (blood, heart, liver, lungs, kidneys, bone, thyroid, muscle, brain and tumor) are excised, rinsed in saline, and blotted dry. The tissues are weighed, placed into 12×75 mm glass vials, the radioactivity determined with a gamma counter, and the percent dose/gram calculated. Total activities of blood and muscle are calculated by assuming that they account for 7% and 40% of the total body mass, respectively. Examples of the tissue distribution results in shown in Tables 3-7.

TABLE 3
Anti-2-[¹⁸F]FACPC Enantiomer 1 in A549 Human Lung Cancer Cells

| | blood | heart | lung | liver | pancreas | spleen | kidney | muscle | brain | tumor | bone |
|---|---|---|---|---|---|---|---|---|---|---|---|
| 15 min | | | | | | | | | | | |
| ave | 3.98 | 3.78 | 4.09 | 4.01 | 57.83 | 12.52 | 8.0 | 2.47 | 0.81 | 9.77 | 3.08 |
| s.d. | 0.69 | 0.69 | 0.41 | 1.24 | 14.36 | 4.46 | 0.70 | 0.89 | 0.16 | 3.08 | 1.08 |
| 60 min | | | | | | | | | | | |
| ave | 1.59 | 2.49 | 2.07 | 2.16 | 27.09 | 4.13 | 4.02 | 2.43 | 0.81 | 7.16 | 2.43 |
| s.d. | 0.33 | 0.44 | 0.57 | 0.54 | 11.16 | 2.09 | 0.88 | 0.74 | 0.17 | 1.95 | 0.95 |

TABLE 4
Anti 2-[¹⁸F]FACPC Enantiomer 2 in A549 Human Lung Cancer Cells

| | blood | heart | lung | liver | pancreas | spleen | kidney | muscle | brain | tumor | bone |
|---|---|---|---|---|---|---|---|---|---|---|---|
| 15 min | | | | | | | | | | | |
| ave | 3.68 | 3.18 | 4.75 | 2.86 | 40.68 | 9.37 | 10.81 | 2.07 | 0.53 | 8.13 | 2.50 |
| s.d. | 0.44 | 0.23 | 0.42 | 0.25 | 2.83 | 4.31 | 1.31 | 0.57 | 0.07 | 1.67 | 0.48 |
| 60 min | | | | | | | | | | | |
| ave | 1.17 | 1.82 | 1.77 | 1.22 | 16.42 | 2.95 | 3.73 | 1.78 | 0.58 | 5.78 | 1.77 |
| s.d. | 0.22 | 0.16 | 0.31 | 0.24 | 4.52 | 0.88 | 0.60 | 0.53 | 0.04 | 1.18 | 0.12 |

TABLE 5
Anti-2-[¹⁸F]FACPC Enantiomer 1 in DU145 Human Prostate Cancer Cells

| | blood | heart | lung | liver | pancreas | spleen | kidney | muscle | brain | tumor | bone |
|---|---|---|---|---|---|---|---|---|---|---|---|
| 15 min | | | | | | | | | | | |
| ave | 3.69 | 3.76 | 4.38 | 4.34 | 47 | 7.4 | 8.83 | 2.23 | 0.70 | 3.82 | 2.89 |
| s.d. | 0.22 | 0.36 | 0.23 | 0.27 | 2.8 | 1.4 | 1.19 | 0.39 | 0.09 | 0.69 | 0.32 |
| 30 min | | | | | | | | | | | |
| ave | 2.44 | 2.71 | 2.78 | 2.74 | 28 | 6.70 | 5.36 | 2.31 | 0.49 | 3.54 | 3.06 |
| s.d. | 0.18 | 0.57 | 0.23 | 0.27 | 4.1 | 2.44 | 0.36 | 0.38 | 0.08 | 0.45 | 0.73 |
| 60 min | | | | | | | | | | | |
| ave | 1.7 | 2.2 | 1.91 | 1.96 | 22 | 2.79 | 4.07 | 2.15 | 0.62 | 3.07 | 2.58 |
| s.d. | 0.21 | 0.27 | 0.33 | 0.49 | 6.2 | 0.63 | 0.82 | 0.24 | 0.07 | 0.56 | 0.21 |

TABLE 6
Racemic anti-2-[¹⁸F]FACBC in DU145 Human Prostate Cancer Cells

| | blood | heart | lung | liver | pancreas | spleen | kidney | muscle | brain | tumor | bone | Minutes |
|---|---|---|---|---|---|---|---|---|---|---|---|---|
| Ave | 2.80 | 2.07 | 3.35 | 2.32 | 29.02 | 4.38 | 6.82 | 0.83 | 0.17 | 2.95 | 1.06 | 15 (n = 5) |
| s.d. | 0.81 | 0.67 | 1.38 | 0.73 | 14.70 | 2.99 | 2.89 | 0.33 | 0.05 | 1.20 | 0.57 | |
| Ave. | 1.86 | 1.82 | 2.32 | 2.09 | 29.29 | 4.74 | 4.47 | 1.00 | 0.16 | 3.54 | 1.63 | 30 (n = 5) |
| s.d. | 0.08 | 0.21 | 0.20 | 0.34 | 5.78 | 1.24 | 0.25 | 0.26 | 0.02 | 0.81 | 0.56 | |
| Ave. | 0.80 | 1.32 | 1.18 | 1.23 | 15.01 | 2.85 | 1.82 | 1.15 | 0.18 | 2.57 | 2.22 | 60 (n = 5) |
| s.d. | 0.13 | 0.18 | 0.29 | 0.16 | 3.05 | 0.87 | 0.59 | 0.28 | 0.03 | 0.47 | 1.12 | |
| Ave. | 0.39 | 0.82 | 0.46 | 0.43 | 5.92 | 0.87 | 0.78 | 0.72 | 0.13 | 1.85 | 0.66 | 120 (n = 5) |
| s.d. | 0.08 | 0.20 | 0.14 | 0.12 | 1.79 | 0.34 | 0.24 | 0.12 | 0.03 | 0.19 | 0.10 | |

TABLE 7
Racemic anti-2-[¹⁸F]FACBC in A549 Human Lung Cancer Cells

| | blood | heart | lung | liver | pancreas | spleen | kidney | muscle | brain | tumor | bone | Minutes |
|---|---|---|---|---|---|---|---|---|---|---|---|---|
| Ave | 3.39 | 2.71 | 4.23 | 3.15 | 48.45 | 7.91 | 10.12 | 1.38 | 0.27 | 5.11 | 2.07 | 15 (n = 5) |
| s.d. | 0.23 | 0.26 | 0.57 | 0.67 | 7.69 | 2.12 | 1.01 | 0.19 | 0.02 | 0.85 | 0.43 | |
| Ave. | 2.43 | 2.24 | 3.03 | 2.70 | 44.17 | 5.03 | 5.48 | 1.44 | 0.26 | 4.29 | 1.64 | 30 (n = 5) |
| s.d. | 0.72 | 0.21 | 0.29 | 0.55 | 4.28 | 1.62 | 1.46 | 0.33 | 0.05 | 0.94 | 0.18 | |
| Ave. | 1.09 | 1.41 | 1.40 | 1.47 | 20.64 | 2.86 | 2.25 | 0.90 | 0.16 | 3.04 | 1.02 | 60 (n = 5) |
| s.d. | 0.18 | 0.36 | 0.32 | 0.48 | 3.50 | 1.03 | 0.36 | 0.18 | 0.03 | 0.59 | 0.14 | |
| Ave. | 0.41 | 0.86 | 0.56 | 0.59 | 9.80 | 1.48 | 1.03 | 0.77 | 0.15 | 2.30 | 0.62 | 120 (n = 4) |
| s.d. | 0.03 | 0.02 | 0.07 | 0.04 | 1.19 | 0.44 | 0.13 | 0.07 | 0.01 | 0.41 | 0.08 | |

Tables 3-7 show the results of the biodistribution studies with the separate enantiomers of anti-2-[¹⁸F]FACPC and racemic anti-2-[¹⁸F]FACBC as a comparison in SCiD mice implanted in their flanks with A549, human lung cancer cells and DU145, human prostate cancer cells. The uptake of radioactivity after injection of anti-2-[¹⁸F]FACPC and racemic anti-2-[¹⁸F]FACBC in the tumors were greater than muscle at time points sampled post injection.

Transport Mechanism of the Radiolabeled Amino Acids in Different Tumor Cell Lines with Different Malignant Phenotypes.

The uptake of all compounds were measured in human and rat cancer cell lines and the dominant transport (“A” and “L”) mechanism of each culture were determined. The human cancer cell lines chosen can be grown in vitro.

Approximately 10⁶ cells were exposed to the [¹⁸F]amino acid candidate (5 μCi) in 3 ml of amino acid-free media±transporter inhibitors (10 mM) for 30 minutes under incubation conditions by the method previously described (McConathy, J.; Martarello, L.; Malveaux, E.; Camp, V.; Bowers, G.; Olson, J.; Goodman, M. (2002) Radiolabeled amino acids for tumor imaging with PET: radiosynthesis and biological evaluation of [¹⁸F]2-amino-3-fluoro-2-methylpropanoic acid and [¹⁸F]3-fluoro-2-methyl-2-(methylamino)-propanoic acid. J. Med. Chem. 45, 2240-2249.). The inhibition studies determined the dominant transport mechanism (L, A) for each candidate PET amino acid for each tumor model. 2-Methylaminoisobutyric acid (MeAlB) and 2-aminobicyclo-[2.2.1]-heptane-2-carboxylic acid (BCH) serve as inhibitors for the “A”, “L” transport systems, respectively.

These studies indicate that both enantiomer 1 and 2 of anti-2-[¹⁸F]FACPC and racemic anti-2-[¹⁸F]FACBC compounds are selectively taken up in tumor cells. Enantiomer 1 of anti-2-[¹⁸F]FACPC shows greater uptake than enantiomer 2 in both in vitro in cancer cells (Table 2) and in vivo in A549 cells implanted subcutaneously in the flanks of SCID mice (Tables 3 and 4). A comparison of enantiomer 1 of anti-2-[¹⁸F]FACPC with racemic anti-2-[¹⁸F]FACBC show that enantiomer 1 of anti-2-[¹⁸F]FACPC shows significantly higher in vivo uptake than racemic 2-[¹⁸F]FACBC in DU 145 and A549 human cells implanted subcutaneously in the flanks of SCID mice.

The present invention also includes stereoisomers as well as optical isomers, e.g. mixtures of enantiomers as well as individual enantiomers and diastereomers which arise as a consequence of structural asymmetry.

The compounds described herein may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When the compounds of the invention are to be used as imaging agents, they must be labeled with suitable radioactive halogen isotopes such as ¹²³I, ¹³¹I, ¹⁸F, ⁷⁶Br, and ⁷⁷Br. The radiohalogenated compounds of this invention can easily be provided in kits with materials necessary for imaging a tumor. For example, a kit can contain a final product labeled with an appropriate isotope (e.g. ¹⁸F) ready to use for imaging or an intermediate compound and a label (e.g. K[¹⁸F]F) with reagents (e.g. solvent, deprotecting agent) such that a final product can be made at the site or time of use.

In the first step of the present method of imaging, a labeled compound of formula I is introduced into a tissue or a patient in a detectable quantity. The compound is typically part of a pharmaceutical composition and is administered to the tissue or the patient by methods well known to those skilled in the art. For example, the compound can be administered either orally, rectally, parenterally (intravenous, by intramuscularly or subcutaneously), intracistemally, intravaginally, intraperitoneally, intravesically, locally (powders, ointments or drops), or as a buccal or nasal spray.

In an imaging method of the invention, the labeled compound is introduced into a patient in a detectable quantity and after sufficient time has passed for the compound to become associated with tumor tissues or cells, the labeled compound is detected noninvasively inside the patient. In another embodiment of the invention, a labeled compound of formula I is introduced into a patient, sufficient time is allowed for the compound to become associated with tumor tissues, and then a sample of tissue from the patient is removed and the labeled compound in the tissue is detected apart from the patient. Alternatively, a tissue sample is removed from a patient and a labeled compound of formula I is introduced into the tissue sample. After a sufficient amount of time for the compound to become bound to tumor tissues, the compound is detected. The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The administration of the labeled compound to a patient can be by a general or local administration route. For example, the labeled compound may be administered to the patient such that it is delivered throughout the body. Alternatively, the labeled compound can be administered to a specific organ or tissue of interest.

Those skilled in the art are familiar with determining the amount of time sufficient for a compound to become associated with a tumor. The amount of time necessary can easily be determined by introducing a detectable amount of a labeled compound of formula I into a patient and then detecting the labeled compound at various times after administration.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. PET and SPECT are preferred when the compounds of the invention are used as tumor imaging agents. The label that is introduced into the compound will depend on the detection method desired. For example, if PET is selected as a detection method, the compound must possess a positron-emitting atom, such as ¹¹C or ¹⁸F.

The radioactive diagnostic agent should have sufficient radioactivity and radioactivity concentration which can assure reliable diagnosis. For instance, in case of the radioactive metal being technetium-99m, it may be included usually in an amount of 0.1 to 50 mCi in about 0.5 to 5.0 ml at the time of administration. The amount of a compound of formula may be such as sufficient to form a stable chelate compound with the radioactive metal.

The inventive compound as a radioactive diagnostic agent is sufficiently stable, and therefore it may be immediately administered as such or stored until its use. When desired, the radioactive diagnostic agent may contain any additive such as pH controlling agents (e.g., acids, bases, buffers), stabilizers (e.g., ascorbic acid) or isotonizing agents (e.g., sodium chloride). The imaging of a tumor can also be carried out quantitatively using the compounds herein so that a therapeutic agent for a given tumor can be evaluated for its efficacy.

Preferred compounds for imaging include a radioisotope such as ¹²³I, ¹²⁴I, ¹²⁵I, ¹³¹I, ¹⁸F, ⁷⁶Br, ⁷⁷Br or ¹¹C.

The synthetic schemes described herein represent exemplary syntheses of preferred embodiments of the present invention. However, one of ordinary skill in the art will appreciate that starting materials, reagents, solvents, temperature, solid substrates, synthetic methods, purification methods, analytical methods, and other reaction conditions other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

When a group of substituents is disclosed herein, it is understood that all individual members of that group and all subgroups, including any isomers and enantiomers of the group members, are intended to be individually included. When a Markush group or other grouping is used herein, all individual members of the group and all combinations and subcombinations possible of the group are intended to be individually included in the disclosure. When a compound is described herein such that a particular isomer or enantiomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions, those that are appropriate for preparation of salts of this invention for a given application.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All references cited herein are hereby incorporated by reference to the extent that there is no inconsistency with the disclosure of this specification. In particular, U.S. Pat. Nos. 5,817,776, 5,808,146, and WO 03/093412 are cited herein and incorporated by reference herein to provide examples of the amino acid analogs that can be made using the invention and the detailed synthetic methods. Some references provided herein are incorporated by reference to provide details concerning sources of starting materials, additional starting materials, additional reagents, additional methods of synthesis, additional methods of analysis and additional uses of the invention.

We claim:

1. An amino acid analog having the general formula: 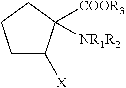
wherein R1 and R2 are each independently selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, haloalkynyl, Tc-99m and Re chelates;
R3 is selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, and haloalkynyl;
X is selected from the group consisting of halogen, haloalkyl, halocycloalkyl, halocycloalkenyl, halocycloalkynyl, haloacyl, haloaryl, haloheteroaryl, haloalkenyl, haloalkynyl, Tc-99m chelate and Re chelate, where halo or halogen in X is selected from the group consisting of F, Cl, Br, I, At, F-18, Br-76, I-123, I-124, or a pharmaceutically acceptable salt thereof.

2. The amino acid analog of claim 1 wherein X is selected from the group consisting of halo, C1-C6 haloalkyl, C1-C6 halocycloalkyl, C1-C6 halocycloalkenyl, C1-C6 halocycloalkynyl, haloacyl, haloaryl, haloheteroaryl, C1-C6 haloalkenyl, and C1-C6 haloalkynyl.

3. The amino acid analog of claim 2 wherein R1, R2 and R3 are each independently of each other selected from the group consisting of hydrogen, C1-C6 alkyl, C1-C6 alkenyl and C1-C6 alkynyl.

4. The amino acid analog of claim 2 or 3 wherein X is selected from the group consisting of halogen, C1-C6 haloalkyl, C1-C6 haloalkenyl, and C1-C6 haloalkynyl, wherein halo is either ¹⁸F or ¹²³I.

5. The amino acid analog of claim 4 wherein X is halogen or C1-C4 haloalkyl and R₁, R₂, and R₃ are independently of each other hydrogen or C1-C4 alkyl.

6. The amino acid analog of claim 1 which is (1S,2S) anti-2-[¹⁸F]FACPC.

7. The amino acid analog of claim 1 which is (1R,2R) anti-2-[¹⁸F]FACPC.

8. The amino acid analog of claim 1 having the following formula: 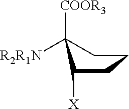
wherein R1 and R2 are each independently selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, haloalkynyl, Tc-99m and Re chelates;
R3 is selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, and haloalkynyl;
X is selected from the group consisting of halogen, haloalkyl, halocycloalkyl, halocycloalkenyl, halocycloalkynyl, haloacyl, haloaryl, haloheteroaryl, haloalkenyl, haloalkynyl, Tc-99m chelate and Re chelate, where halo or halogen in X is selected from the group consisting of F, Cl, Br, I, At, F-18, Br-76, I-123, I-124, or a pharmaceutically acceptable salt thereof.

9. The amino acid analog of claim 8, wherein R1, R2 and R3 are H and X is ¹⁸F.

10. The amino acid analog of claim 1 having the following formula: 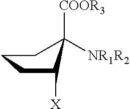
wherein R1 and R2 are each independently selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, haloalkynyl, Tc-99m and Re chelates;
R3 is selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, and haloalkynyl;
X is selected from the group consisting of halogen, haloalkyl, halocycloalkyl, halocycloalkenyl, halocycloalkynyl, haloacyl, haloaryl, haloheteroaryl, haloalkenyl, haloalkynyl, Tc-99m chelate and Re chelate, where halo or halogen in X is selected from the group consisting of F, Cl, Br, I, At, F-18, Br-76, I-123, I-124, or a pharmaceutically acceptable salt thereof.

11. The amino acid analog of claim 10, wherein R1, R2 and R3 are H and X is ¹⁸F.

12. A method of synthesizing an amino acid analog according to claim 1 comprising the step of reacting a compound of formula III with reagents to yield the amino acid analog of formula I, wherein formula III is: 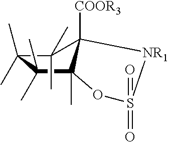
wherein
R1 is selected from the group consisting of H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, haloalkynyl, Tc-99m and Re chelates; and
R3 is selected from the group consisting of: H, alkyl, haloalkyl, cycloalkyl, halocycloalkyl, cycloalkenyl, halocycloalkenyl, cycloalkynyl, halocycloalkynyl, acyl, haloacyl, aryl, haloaryl, heteroaryl, haloheteroaryl, alkenyl, haloalkenyl, alkynyl, and haloalkynyl.

13. The method of claim 12 wherein the amino acid analog is anti-2-[¹⁸F]FACPC.

14. A diagnostic composition for imaging a tumor, comprising a radiolabeled compound of claim 1, and a pharmaceutically acceptable carrier.

15. The diagnostic composition of claim 14 wherein the labeled compound is (1S,2S) anti-2-[¹⁸F]FACPC.

16. The diagnostic composition of claim 14 wherein the labeled compound is (1R,2R) anti-2-[¹⁸F]FACPC.

17. A method of tumor imaging by positron emission tomography or single photon emission computed tomography, comprising: a) administering to a subject suspected of having a tumor an image-generating amount of a labeled compound of claim 1; b) allowing sufficient time for the labeled compound to become associated with the tumor; and c) measuring the distribution of the labeled compound in the subject by PET or SPECT.

18. The method of claim 17 wherein the labeled compound is (1S,2S) anti-2-[¹⁸F]FACPC.

19. The method of claim 17 wherein the labeled compound is (1R,2R) anti-2-[¹⁸F]FACPC.
